(19) United States
(12) Patent Application Publication
(10) Pub. No.: US 2009/0264863 A1
(43) Pub. Date: Oct. 22, 2009

(54) Articulating Tip Tetherless Catheter System

(75) Inventor: Eliot Frank Bloom, Hopkinton, NH (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/365,983

(22) Filed: Feb. 5, 2009

Related U.S. Application Data
(60) Provisional application No. 61/046,839, filed on Apr. 22, 2008.

(51) Int. Cl. A61M 25/098

(52) U.S. Cl. 604/524

(57) ABSTRACT

A catheter system includes a concentrically arranged proximal catheter and an inner catheter; a conical tip attached to a distal end of the inner catheter; and at least one articulating assembly disposed on the catheter. The articulating assembly includes a distal ring attached to the inner catheter adjacent a proximal end of the conical tip and a proximal ring having a wedge shaped cross section attached to the distal end of the proximal catheter. A method for navigating a vascular system includes providing a catheter system, navigating the distal end of the catheter through the vascular system; determining a change in direction of the distal end of the catheter; and manipulating the at least one articulating assembly so that a distal ring and a proximal ring are rotated relative to each other and the angle of the distal tip relative to a long axis of the catheter has been altered

### RELATED APPLICATIONS

This application claims the benefit of, and priority to, U.S. Provisional Patent Application 61/046,839 filed on Apr. 22, 2008, to Eliot Bloom, entitled Articulating Tip Tetherless Catheter System, the entirety of which is incorporated herein by reference.

### FIELD OF INVENTION

This invention relates generally to medical devices and procedures, and more particularly to a device and system for delivering an implantable medical device to a location in a vascular system.

### BACKGROUND OF THE INVENTION

Heart valves, such as the mitral, tricuspid, aortic and pulmonary valves, are sometimes damaged by disease or by aging, resulting in problems with the proper functioning of the valve. Heart valve problems generally take one of two forms: stenosis, in which a valve does not open completely or the opening is too small, resulting in restricted blood flow; or insufficiency, in which blood leaks backward across a valve when it should be closed.

Previously, valve repair or replacement required open-heart surgery with its attendant risks, expense, and extended recovery time. Open-heart surgery also requires cardiopulmonary bypass with risk of thrombosis, stroke, and infarction. More recently, flexible valve prostheses and various delivery devices have been developed so that replacement valves can be implanted transvenously using minimally invasive techniques.

Recently, implantable heart valves have been developed that can be delivered transvenously using a catheter-based delivery system. These valves comprise a collapsible valve attached to the interior of a tubular frame or stent. The valve can be any of the valve prostheses described above, or it can be any other suitable valve. In the case of valves in harvested vessels, the vessel can be of sufficient length to extend beyond both sides of the valve such that it extends to both ends of the valve support stent.

The valves can also comprise a tubular portion or “stent graft” that can be attached to the interior or exterior of the stent to provide a generally tubular internal passage for the flow of blood when the leaflets are open. The graft can be separate from the valve and it can be made from any suitable biocompatible material including, but not limited to, fabric, a homograft, porcine vessels, bovine vessels, and equine vessels.

The stent portion of the device can be reduced in diameter, mounted on a catheter, and advanced through the circulatory system of the patient. The stent portion can be either self-expanding or balloon expandable. In either case, the stented valve can be positioned at a delivery site, where the stent portion is expanded against the wall of a previously implanted prosthesis, or against the wall of a native vessel or heart chamber to hold the valve firmly in place.

During delivery of these valves, the catheter is maneuvered, until the end of the catheter is positioned in the vicinity of the intended treatment site. The inner tube is then held stationary while the sheath of the delivery catheter is withdrawn. For a self expanding configuration the inner tube prevents the stent-graft from moving back as the sheath is withdrawn.

As the sheath is withdrawn, the stent is gradually exposed from a proximal end to a distal end of the stent-graft, the exposed portion of the stent-graft radially expands so that at least a portion of the expanded portion is in substantially conforming surface contact with a portion of the interior of the lumen (e.g., arterial wall).

In straight anatomies, delivery of an implantable device by catheter is relatively straightforward. However, delivery can be difficult in complex anatomies. Examples of such difficult procedures are catheter delivery of a prosthetic aortic valve catheter delivery of a prosthetic pulmonic valve, or catheter delivery of a prosthetic mitral valve; all of which present a significantly complex route for navigation by a catheter with a relatively large diameter.

Some catheters and endoscopes can be remotely steered. For example, U.S. Pat. No. 5,325,845 suggests a steerable sheath for use in connection with optical catheters. The proximal end of the catheter is provided with a pair of steering knobs which are connected to wires that run along the length of the catheter. Each knob controls a pair of diametrically opposed wires and all four of the wires are attached to the distal tip of the catheter. By appropriate manipulation of either of the control knobs, one can ostensibly control the position of the distal tip of the catheter. By such remote manipulation, the reference claims a physician can move the optical catheter into position to view the desired site. Others have proposed similar uses of cables in endoscopic procedures. For example, U.S. Pat. No. 4,700,693 suggests a design which utilizes steering cables and a number of washers. The steering cables can be remotely manipulated to guide the endoscope through a desired curve.

There are also steerable and formable catheters that can be used to deliver therapeutic devices to a body through lumens in the catheter. U.S. Pat. No. 5,916,147, U.S. Pat. No. 6,544,215, and U.S. Pat. No. 6,991,616 are examples of such catheters. One thing that most steerable catheters have in common is that there must be some lumen or other space in the catheter for the control members that are used to remotely manipulate the catheter sections inside of a patient's body. These control member lumens take up space, which causes an increase in the overall delivery profile. Because catheters used for delivering medical devices such as heart valves and stent grafts can have relatively large crossing profiles to begin with, it would be desirable to provide means to steer such catheters that would not significantly increase the diameter of the catheter.

Thus, it would be desirable to provide devices and systems that will allow navigation through difficult, tortuous, and complex anatomy by relatively large diameter catheters for delivery of implantable devices. It would also be desirable to provide methods for using such devices and systems.

### SUMMARY OF THE INVENTION

The present invention discloses catheter or delivery systems having a selectively rotatable tip section that can be used for assisting in navigation through complex vascular anatomy. The present invention also provides a flexible conical tip portion to further assist in navigation through the vasculature.

One aspect of the invention provides a catheter system for use in a medical procedure. The system comprises an elongate, flexible, generally tubular proximal catheter and an elongate, flexible generally tubular inner catheter; the inner catheter and the proximal catheter each having a proximal section, a distal section, and a central lumen passing therethrough, the inner catheter and the proximal catheter concentrically arrange configuration. The system further includes a conical tip attached to the distal end of the inner catheter; and at least one articulating assembly disposed on the catheter. The articulating assembly comprises a distal ring attached to the inner catheter adjacent a proximal end of the conical tip; and a proximal ring having a wedge shaped cross section attached to the distal end of the proximal catheter.

Another aspect of the invention provides a method for navigating a catheter through a patient's vascular system. The method comprises providing a catheter system according to the invention, navigating the distal end of the catheter through a patient's vascular system; determining a change in direction of the distal end of the catheter; and manipulating the at least one articulating assembly so that the distal ring and the proximal ring are rotated relative to each other and the angle of the distal tip relative to a long axis of the catheter has been altered.

The aforementioned and other features and advantages of the invention will become further apparent from the following detailed description of the presently preferred embodiments, read in conjunction with the accompanying drawings, which are not to scale. The drawings should not be taken to limit the invention to the specific embodiments, but are for explanation and understanding. The detailed description and drawings are merely illustrative of the invention rather than limiting, the scope of the invention being defined by the appended claims and equivalents thereof.

### BRIEF DESCRIPTION OF THE DRAWINGS

FIG. 1 is a schematic interior view of heart showing the interior structure of the heart;

FIG. 2 is a plan view of a heart showing the location of the heart valves;

FIG. 3 shows one embodiment of a catheter tip according to the current invention;

FIGS. 4A and 4B show another embodiment of a catheter tip according to the current invention;

FIGS. 5A, 5B and 6 show more detail of the catheter tip shown in FIGS. 4A and 4B;

FIG. 7 shows another embodiment of a catheter tip according to the current invention;

FIG. 8 shows the catheter having a catheter tip shown in FIG. 7 within the aortic arch according to the current invention; and

FIG. 9 is a flow chart of a method of navigating a vascular system according to the current invention.

### DETAILED DESCRIPTION

The invention will now be described by reference to the figures wherein like numbers refer to like structures. The terms “distal” and “proximal” are used herein with reference to the treating clinician during the use of the catheter system; “distal” indicates an apparatus portion distant from, or a direction away from the clinician and “proximal” indicates an apparatus portion near to, or a direction towards the clinician. The term “magnet” as used herein indicates a material that exerts an attractive or repulsive force on other materials.

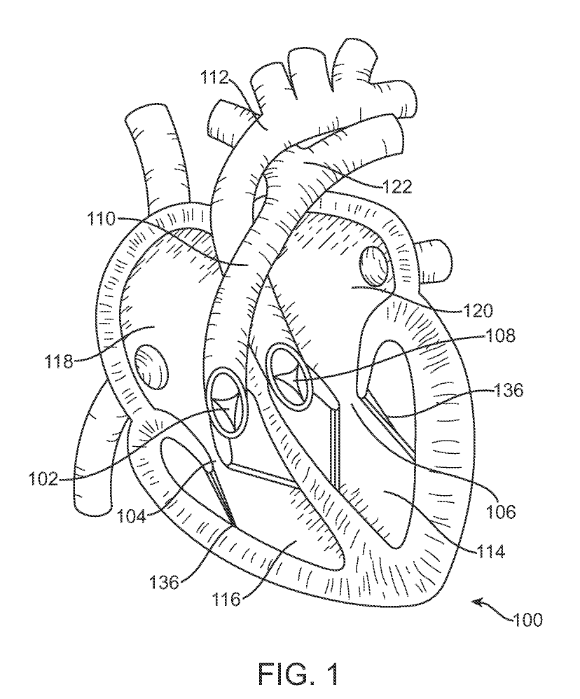

Referring to the drawings, FIG. 1 is a schematic representation of the interior of human heart 100. Human heart 100 includes four valves that work in synchrony to control the flow of blood through the heart. Tricuspid valve 104, situated between right atrium 118 and right ventricle 116, and mitral valve 106, between left atrium 120 and left ventricle 114 facilitate filling of ventricles 116 and 114 on the right and left sides, respectively, of heart 100. Also shown in the figure are chordae tendenae 136, attached to the valve leaflets and papillary muscle.

Aortic valve 108 is situated at the junction between aorta 112 and left ventricle 114 and facilitates blood flow from heart 100, through aorta 112 to the peripheral circulation. Pulmonary valve 102 is situated at the junction of right ventricle 116 and pulmonary artery 110 and facilitates blood flow from heart 100 through the pulmonary artery 110 to the lungs for oxygenation. The four valves work by opening and closing in harmony with each other.

During diastole, tricuspid valve 104 and mitral valve 106 open and allow blood flow into ventricles 114 and 116, and the pulmonic valve and aortic valve are closed. During systole, shown in FIG. 1, aortic valve 108 and pulmonary valve 102 open and allow blood flow from left ventricle 114, and right ventricle 116 into aorta 112 and pulmonary 110, respectively.

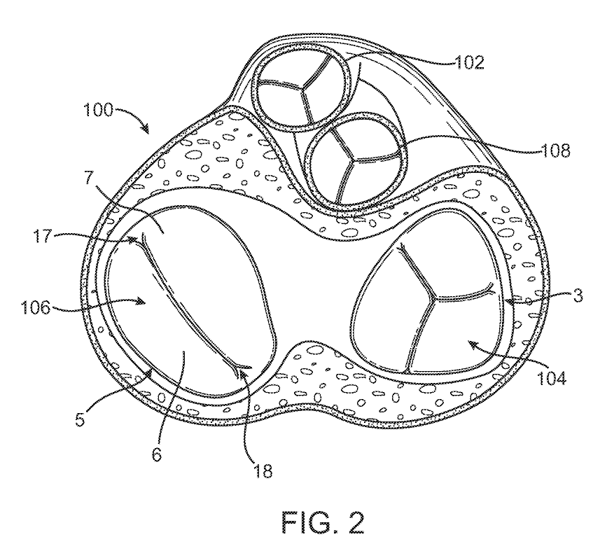

FIG. 2 shows a plan view of a cross-section of heart 100 having tricuspid valve 104 and tricuspid valve annulus 3. Mitral valve 106 is adjacent mitral valve annulus 5. Mitral valve 106 is a bicuspid valve having anterior cusp 7 and posterior cusp 6. Anterior cusp 7 and posterior cusp 6 are often referred to, respectively, as the anterior and posterior leaflets. Also shown in the figure are the posterior commisure 17 and the anterior commisure 18.

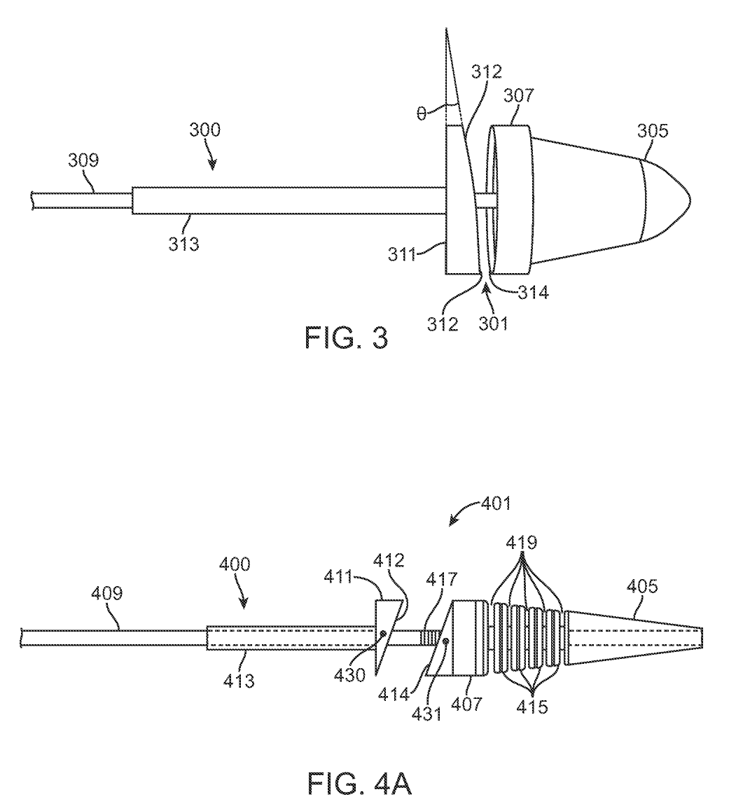

Referring now to FIGS. 3 to 8, there are shown embodiments of delivery systems with articulating tips. The tips of the delivery systems include a series of rings, of which at least one is controlled via rotation of a catheter to which the ring is attached. The angle of the tip can then be precisely controlled by rotation of the rings relative to each other and a subsequent engagement of the rings. If a change of tip angle is required it is accomplished with a simple disengagement of the rings, a rotation of at least one of the rings to the desired angle and then a reengagement of the rings.

Referring now to FIG. 3, there is shown one embodiment of a catheter 300 having an articulating assembly 301. Catheter 300 includes a distal tip 305 with a parabolic shape. The catheter tips of the current invention can be made from a biocompatible polymer and coated with an elastic coating, or they can be made entirely from an elastic material such as and without limitation, silastic, silicone, urethane and the like. The material used to make or coat the tips can be lubricious or the tips can be coated with a hydrophilic material. The tips are attached to, bonded to, or integrally constructed as part of a concentric two catheter system with the tip being at the distal most end of an elongated inner catheter having a guidewire lumen.

Articulating assembly 301 includes distal ring 307 and proximal ring 311. Distal ring 307 is located at the most proximal region of distal tip 305. In one embodiment, distal ring 307 is formed integrally with tip 305. In another embodiment, distal ring 305 is formed apart from distal tip 305 and subsequently attached to the proximal end of distal tip 305 and/or the inner catheter by welding, adhesive or any other manner known in the art. Distal tip 305 is connected to an elongated, generally tubular inner catheter 309. In one embodiment, inner catheter 309 includes a lumen that communicates along the length of the inner catheter 309 to the distal most end of the tip 305. In one embodiment, the inner catheter lumen is a guidewire lumen.

Proximal ring 311 includes a generally wedge shaped cross section. Proximal ring 311 is connected to an elongated, generally tubular proximal catheter 313 such that an angled top (or distal) surface 312 of the proximal ring is facing a bottom (or proximal) surface 314 of distal ring 307. Proximal catheter 313 includes a lumen that communicates along the length of proximal catheter 313 and receives inner catheter 309. As such proximal catheter 313 and inner catheter comprise a concentrically arranged catheter.

In the embodiment depicted in FIG. 3, the wedge shaped proximal ring 311 is made from a magnet, or magnetized material, and at least a portion of the distal ring 307 of tip 305 is made from material that is attracted to the magnet. In other embodiments, the distal ring can be made from a magnet and the proximal ring is made from material that is attracted to magnets. In at least one embodiment, both the distal ring and the proximal ring are made from magnets. The size of the magnet depends on factors such as, but not limited to, the diameter of the ring, the material composing the magnet, the size of the catheter, the desired amount of attractive force between the rings and the particular application for which the catheter is being used. In an example, a ring having a smaller diameter may require a stronger magnet to provide the necessary amount of attraction to attract and hold the opposing ring. The magnetic ring is composed of any suitable biocompatible magnetic material or material capable of being magnetized such as stainless steel and ceramic. In some embodiments, the magnet may be composed of a ferromagnetic material. In these embodiments, the ring is coated or plated with a biocompatible material such as gold or silver.

The angle θ of the wedge for proximal ring 311 can be selected based on the type of treatment for which the catheters including an articulating tip are being used. In some embodiments of the invention, articulating assemblies have proximal rings with angles in the range of 15 to 45 degrees. At least one embodiment of a catheter system according to the current invention has a proximal ring with an angle of 22.5 degrees. At least one embodiment has a proximal ring with an angle less than 15 degrees and at least one other embodiment has a proximal ring with an angle greater than 45 degrees.

The systems of the current invention are constructed such that the inner catheter and proximal catheter can be moved on the longitudinal axis relative to each other. This can be accomplished by securing the proximal catheter in place and moving the inner catheter distally or proximally, securing the inner catheter in place and moving the proximal catheter distally or proximally, or a combination of movement of the inner and proximal catheters. Longitudinal and rotational movement of the inner catheter and the proximal catheter is controlled at a handle (not shown) operably connected to proximal ends of the inner and proximal catheters.

The catheters of the invention can be made from flexible, biocompatible polymeric materials that are suitable for catheter construction. Examples of such material include, but are not limited to, polyurethane, polyethylene, nylon and polytetrafluoroethylene (PTFE). At least one embodiment of the invention can include a reinforced layer of biocompatible material for at least one of the inner catheter or proximal catheter. The material can be any material known by those having ordinary skill in the art to be suitable for constructing catheters, including PEBAX.

Regardless of the material used to construct the catheters, the system is constructed so that the inner catheter is slightly more flexible than the proximal catheter. This can be accomplished in numerous ways, including, but not limited to, making the walls of the inner catheter thinner than the walls of the proximal catheter, and constructing the two catheters from different material.

When a clinician using the system depicted in FIG. 3, wants the catheter to track in a straight line, he or she moves the proximal ring 311 away from the distal tip so that the distal ring 307 is not attracted to the proximal ring. When the clinician wants to articulate the catheter, the proximal ring is moved towards the distal ring until the bottom surface 314 of the distal ring is touching the top surface 312 of the proximal ring. The more flexible inner catheter will allow the distal tip to be articulated at an angle that is reciprocal to the angle of the proximal ring. A clinician can change the angular orientation of the distal tip by withdrawing the proximal ring, rotating the proximal ring relative to the distal ring, and then moving the proximal ring forward to engage it with the distal ring. The catheters can also be rotated together to allow a clinician to fine tune the vector orientation of the distal tip. When the clinician wants the catheter to track in a straight line again, the proximal ring is disengaged from the distal ring and the system is maintained in this unconstrained configuration until articulation is again desired.

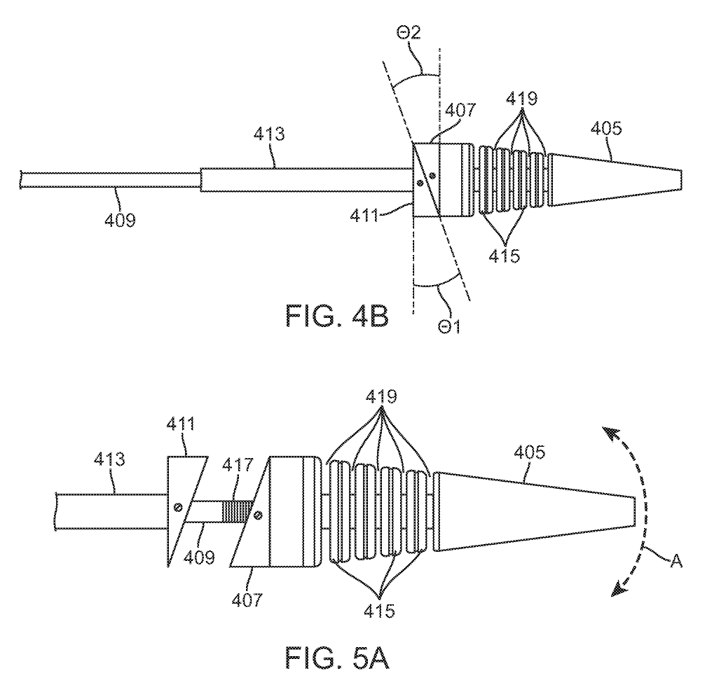

Referring now to FIGS. 4A, 4B, 5A, 5B, and 6, there is shown another embodiment of a catheter system 400 having an articulating tip assembly 401 according to the current invention. The system includes a conical tip 405 constructed or coated with a flexible elastic material. Conical tip 405 includes a plurality of radial slots 419, which define a plurality of radial ribs 415 spaced along a portion of the tip. Articulating tip assembly 401 includes distal ring 407 and proximal ring 411. Distal ring 407, having a generally wedge shaped cross section, is located on the proximal portion of the conical tip 405. Conical tip 405 is attached to an elongated, generally tubular inner catheter 409 that includes a flexible distal portion 417. Proximal ring 411, having a generally wedge shaped cross section, is connected to an elongated, generally tubular proximal catheter 413. The distal ring 407 and the proximal ring 411 are attached to their respective catheters such that the angled surface 412 of the proximal ring 411 is facing the angled surface 414 of the distal ring 407.

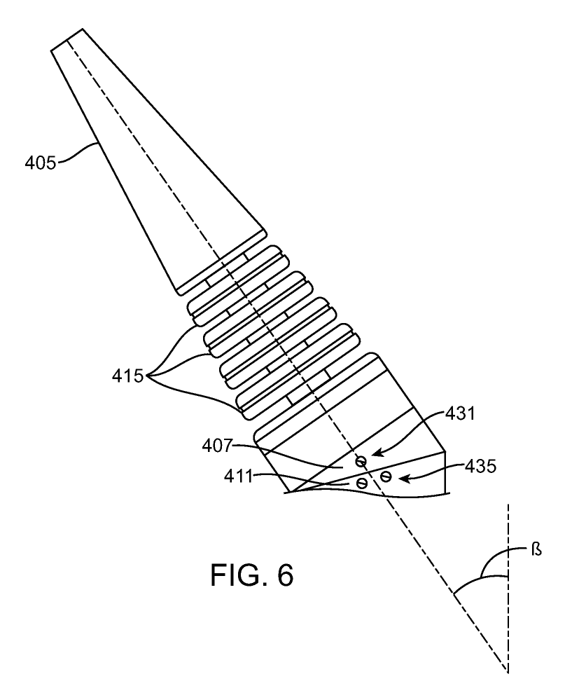

The distal and proximal rings 407, 411 can include radiopaque markers spaced around the perimeters of the rings to assist a clinician in obtaining the desired alignment of the rings. FIG. 4 shows a single marker dot 430 and 431 on each of the distal and proximal rings, respectively. Other markers on other portions of the rings can include a plurality of dots, or different shapes such that when a clinician desires a specific angle, the dots can be lined up appropriately. The radiopaque dot alignment in FIG. 4 shows the catheter configured for straight tracking or standard navigation. As shown in FIG. 6, the depicted embodiment has other dot configurations at other places on the rings. These alternative marker dot configurations are used to place the articulating tip in a variety of positions.

FIG. 4B shows the catheter tip system with the distal 407 and proximal 411 rings engaged. In this embodiment, the distal and proximal rings have identically shaped construction such that when the widest portion of one ring is placed directly adjacent the narrowest potion of the other ring, the catheter system is in a straight tracking configuration. Thus, the angle θ2 of the wedge for the distal ring 407 is the same as the angle θ1 of the wedge of the proximal ring 411. Because both the distal and proximal rings are wedge shaped, a clinician can achieve an angle up to (90−θ) along any desired tracking direction by rotating the distal and proximal rings relative to each other. Embodiments of the invention have rings with angles in the range of 15 to 90 degrees. At least one embodiment of a catheter system according to the current invention has rings with an angle of 45 degrees. At least one embodiment of a catheter system according to the current invention has rings with an angle of 22.5 degrees. At least one embodiment has rings with an angle less than 15 degrees.

In at least one embodiment having a wedge shaped distal and proximal ring, the wedge angle θ2 of the distal ring 407 is not equal to the wedge angle θ1 of the proximal ring 411, which allows for more aggressive articulation of the tip portion of the catheter. At least one such embodiment has either a distal or proximal ring with an angle greater than 45 degrees.

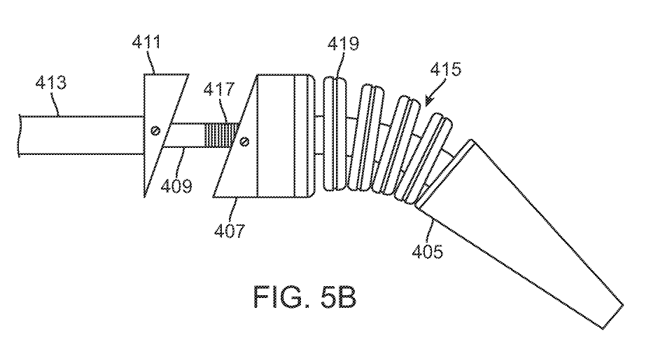

Referring to FIGS. 5A and 5B, the inner catheter 409 contains distal portion 417 that is constructed to be more flexible than the remainder of the inner catheter. In one embodiment, distal portion 417 is accomplished by spiral cutting the distal portion of the inner catheter. In another embodiment, distal portion 417 is accomplished by attaching a spiral cut hypo-tube or a tightly wound helical wire spring as a portion of the inner catheter. The flexible distal portion 417 can be coated or have an outer layer to seal that portion of the catheter from fluid infiltration. The flexible distal portion 417 of the inner catheter 409 allows the tip to quickly articulate based on the orientation of the distal and proximal rings relative to each other. Other embodiments with similar tips can be constructed with an inner catheter having suitable flexibility to articulate without a flexible distal portion as depicted in FIGS. 5A and 5B.

The plurality of radial slots 419 and ribs 415 of top 405 allow the catheter tip to bend in the direction of arrow A. The ability of catheter tip 405 to bend in this manner enables the tip to deflect off of obstacles as the catheter is advanced through a patient's vascular system. The slots also allow a degree of articulation of the tip without changing the orientation of the distal and proximal rings relative to each other. The slots allow the tip to articulate until the outer edges of the ribs can no longer be compressed (between each other or the non-slotted portions of the tip). FIG. 5B illustrates tip 405 in a bent configuration. The tip can be formed or otherwise constructed with a plurality of radial slots 419 and ribs 415, or the slots can be cut after the tip is made. The depth of the slots, the width of the slots and ribs, and the number of slots and ribs will affect the degree of flexibility of the tip. Wide and deep slots will cause the tip to be more flexible than a similar number of narrow or shallower slots. More slots and ribs will cause greater flexibility in the tip also. In at least one embodiment, the tip has five slots that are each at least 0.05 inches wide and extend radially inward to just above the outer surface of the inner catheter.

FIG. 6 shows the device with the proximal ring 411 and the distal ring 407 oriented such that the widest part of the proximal ring 411 is directly adjacent to the widest part of the distal ring 407. When the rings are oriented in such a manner the tip angle from normal β can be determined by subtracting the angle θ of the wedge shaped proximal ring 411 from 90 degrees. FIG. 6 also shows the proximal ring having two radiopaque marker dots 435 that will align with the single marker dot 431 on the distal ring when the distal and proximal rings are engaged as shown in FIG. 6.

When a clinician, using the system depicted in FIGS. 4A-6, wants the catheter to track in a straight line, he or she simply rotates the system such that the widest portion of one ring is placed directly adjacent the narrowest portion of the other ring and any appropriate radiopaque markers are aligned. The clinician then allows the distal ring and proximal ring to engage and begins using the catheter. When the clinician wants to articulate the catheter, the proximal ring and distal ring are separated and rotated relative to each other until desired rotation is achieved and any appropriate markers are aligned. The distal and proximal rings are then engaged and the tip is articulated to a desired angle. The vector orientation of the tip can be fine tuned by rotating the entire catheter system and navigation can resume. When straight line navigation is desired, the rings can be rotated such that the widest portion of one ring is placed directly adjacent the narrowest portion of the other ring and any appropriate radiopaque markers are aligned. This process can be repeated as many times as necessary until the distal tip of the catheter has been navigated to its desired location in the patient's body.

At least one embodiment of the invention includes an expandable medical device, such as a stent or a stent mounted heart valve, disposed on the outermost catheter of the system near the distal end. In at least one embodiment, the system includes a delivery sheath disposed over an expandable medical device. In at least one embodiment, the expandable device is self expanding. In at least one embodiment, the expandable device is balloon expandable. In one embodiment, a stent is disposed on an outer surface of a proximal catheter such as proximal catheter 313, 413. In one embodiment, the stent is a self expanding stent and the system includes a sheath to cover and restrain the stent during delivery to the treatment site.

Embodiments of the devices disclosed or discussed herein can include materials having a high X-Ray attenuation coefficient (radiopaque material) such that the radiopaque material may be visualized using remote visualization techniques. The material can be placed or located on the devices in a manner that would be readily apparent to one of ordinary skill in the art. In one embodiment of the current invention, the catheter and the distal tip each have bands of radiopaque material spaced along a portion thereof. Examples of suitable radiopaque material include, but are not limited to gold, tungsten, silver, iridium, platinum, barium sulfate and bismuth sub-carbonate.

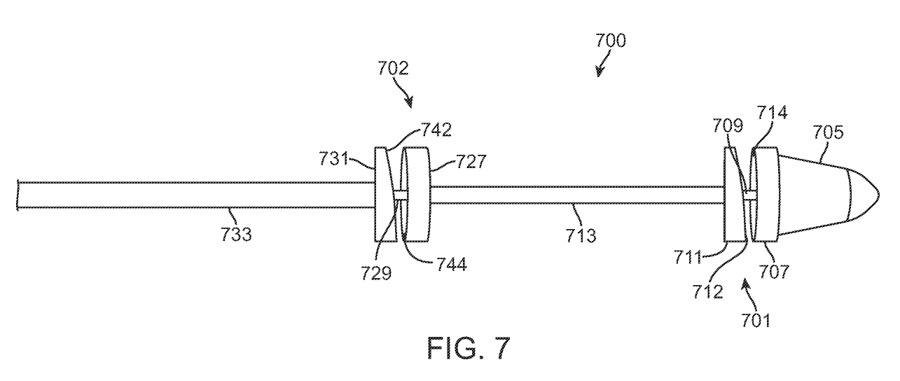

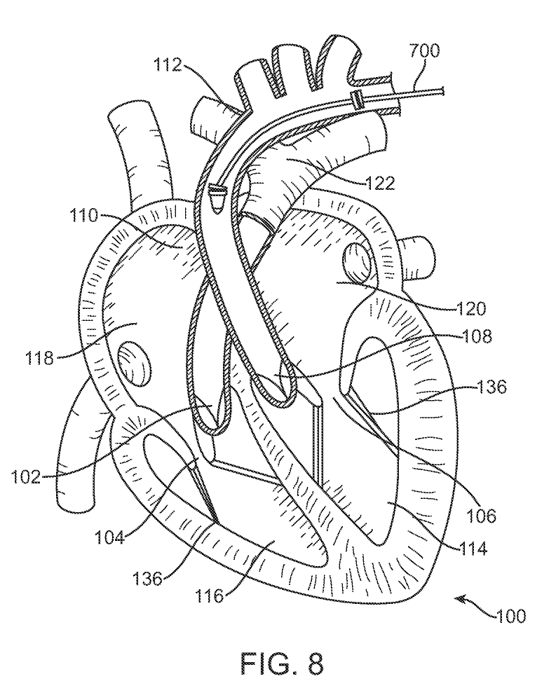

While the invention is described above in terms of being used to control the distal tips of catheters, the invention can also be used to articulate any segment of a catheter. FIGS. 7 and 8 show another embodiment of a catheter system 700 having multiple articulating assemblies 701 and 702. In this embodiment, articulating assembly 701 is the same as or similar to articulating assembly 301, shown in FIG. 3. Articulating assembly 701 includes distal ring 707 located at the most proximal region of distal tip 705. Distal tip 705 may be the same as tip 305 or 405 shown in FIGS. 4A to 6. Distal tip 705 is connected to an elongated, generally tubular inner catheter 709.

Proximal ring 711 includes a generally wedge shaped cross section. Proximal ring 711 is connected to an elongated, generally tubular proximal catheter 713 such that an angled top (or distal) surface 712 of the proximal ring is facing a bottom (or proximal) surface 714 of distal ring 707.

Articulating assembly 702 is spaced apart from, and proximal to, articulating assembly 701 a predetermined distance. The distance that the articulating assemblies are separated may be based on the particular application and/or the particular anatomy through which the catheter system must navigate. Articulating assembly 702 includes distal ring 727 connected to an elongated, generally tubular catheter 729. In this embodiment catheter 729 is a second proximal catheter having a longitudinal lumen for receiving proximal catheter 713. Articulating assembly 702 also includes proximal ring 731 having a generally wedge shaped cross section. Proximal ring 731 is connected to an elongated, generally tubular third proximal catheter 733 such that an angled top (or distal) surface 742 of the proximal ring is facing a bottom (or proximal) surface 744 of distal ring 727. In this embodiment, the catheters of system 700 are concentrically arranged and have proximal ends attached to a control handle. The movement of catheters 709, 713, 729 and 733 are controlled via the handle. Catheters 709, 713, 729 and 733 can be moved independently in relation to each other in a manner the same as or similar to that described above for catheters 300 and 400.

FIG. 8 illustrates catheter system 700 navigating through the aortic arch of heart 100. It will be appreciated that the use of system 700 allows the clinician to use a catheter having a distal tip that is shorter than those of the prior art when delivering a medical device such as a replacement valve over and through the aortic arch. As will also be appreciated by one with ordinary skill in the art, the ability of a catheter to articulate at the tip as well as a position apart from the tip is useful for navigating anatomies having multiple bends and curves or is otherwise tortuous.

Those with ordinary skill in the art will appreciate that articulating assemblies may be placed at positions along a catheter other than those illustrated in FIGS. 3-8. The series of rings disclosed herein could be mounted further back from the tip such that they could be used to articulate any segment of a catheter. In one embodiment, a catheter includes an articulating assembly a predetermined distance away from the distal tip. Still other embodiments have more than two articulating assemblies disposed along the length of the catheter as determined by the particular application and/or the anatomy of the patient's vascular system. Catheters with multiple articulating assemblies also provide a clinician with the ability to bend the catheter in more than one direction as well as more than one plane within the patient's vasculature.

Using the device of the current invention a clinician is now able to articulate the distal tip of a delivery system without any form of tethers or cables. The invention is significant because it provides for the simple articulation of a full 360 degrees without the restriction and additional foot print of a conventional tether based system. Thus, when using the tip disclosed herein, a clinician is no longer restricted to the allowable axis of a single tether or the compound angle generated from a multiple tether articulating system.

The use of this tip would benefit clinicians trying to track a delivery system through the chambers of the heart for structural heart repair, valvuloplasty, ICD/pacemaker lead delivery, etc. In one embodiment, the usage of this system would be very similar to traditional replacement valve delivery systems, in that it rides over a guidewire and it can have an outer sheath to protect the device. In other embodiments, the catheters can be tracked through the system without the use of a guidewire. The current invention would provide a clinician with an advantage when trying to navigate a delivery system past anatomical features (e.g. valve annulus, leaflets, chordae, aortic arch, etc).

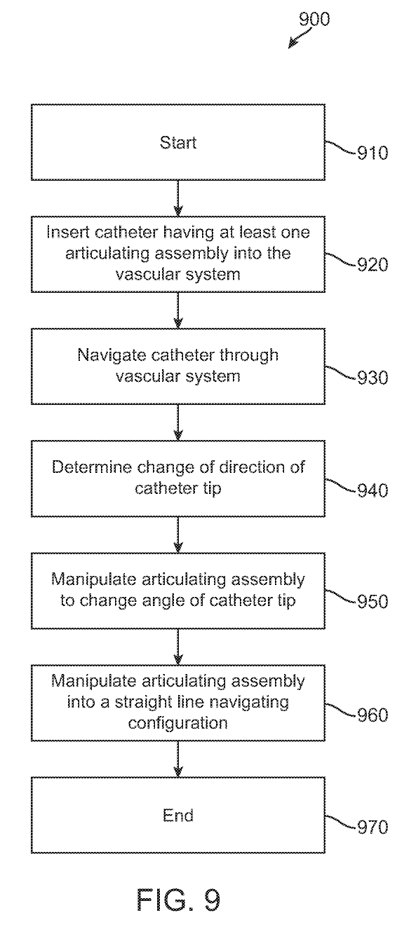

FIG. 9 is a flow chart of one embodiment of a method 900 for navigating a vascular system using a catheter having at least one articulating assembly according to the present invention. Method 900 begins at 910. Throughout the method, a clinician can visualize the navigation of the catheter through the vascular system via any method known to those with skill in the art.

A catheter system, such as systems 300, 400 and 700, having at least one articulating assembly is provided and inserted into a patient's vascular system (Block 920). The catheter system may include a medical device, such as a stent or stented valve, disposed at a distal end of the catheter. A distal end of the catheter is navigated toward a treatment site (Block 930). At block 940, a clinician determines a desired change in direction of the advancing distal end of the catheter. The change in direction may be due to a change in direction of the vascular pathway or a determination that an obstruction lies in the pathway of the distal end of the catheter. Based on this determination, the articulating assembly is manipulated to move a distal ring and a proximal ring relative to each other to thereby change the angle of the distal tip relative to the long axis of the catheter (Block 950). Once the tip angle and direction of the distal tip is achieved, the navigation of the distal end of the catheter continues (Block 960) with the catheter moving in the determined direction or around the obstruction in a straight line navigation configuration. The articulating assembly is returned to a straight line navigation configuration if desired by the clinician. In one embodiment, the articulating assembly is returned to the navigating configuration by moving the distal ring apart from the proximal ring. The articulating assembly may be engaged to change the tip direction as often is necessary to place the distal tip at the treatment site. Navigation of the catheter tip continues until the treatment site is reached. Method 900 ends at Block 970.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A catheter system for use in a medical procedure comprising:
an elongate, flexible, generally tubular proximal catheter;
an elongate, flexible generally tubular inner catheter;
the inner catheter and the proximal catheter each having a proximal section, a distal section, and a central lumen passing therethrough, the inner catheter and the proximal catheter concentrically arrange configuration;
a conical tip attached to the distal end of the inner catheter;
at least one articulating assembly disposed on the catheter, the articulating assembly comprising:
a distal ring attached to the inner catheter; and
a proximal ring having a wedge shaped cross section attached to the distal end of the proximal catheter.

2. The catheter system of claim 1 wherein distal ring also has a wedge shaped cross section.

3. The catheter system of claim 1 wherein the at least one articulating assembly is adjacent a proximal end of the conical tip.

4. The catheter system of claim 1 wherein one of the distal ring or the proximal ring is made from a magnet and the other of the distal ring or proximal ring is made from a material that is attracted to a magnet.

5. The catheter of claim 1 wherein both the distal ring and the proximal ring are made from magnets.

6. The catheter system of claim 1 wherein the catheter has a long axis and the proximal and distal rings are rotatable relative to each other.

7. The catheter system of claim 1 wherein the distal tip further has a plurality of radial slots and a plurality of radial ribs along a portion thereof.

8. The catheter system of claim 1 where in the shape of the distal tip is either conical or parabolic.

9. The catheter system of claim 1 further comprising at least one radiopaque marker on the distal ring and at least one radiopaque marker on the proximal ring.

10. The catheter system of claim 1 wherein the at least one articulating assembly is disposed on the catheter a predetermined distance from a proximal end of the conical tip.

11. The catheter system of claim 1 wherein the at least one articulating assembly comprises a first articulating assembly disposed adjacent the distal tip of the catheter and a second articulating assembly spaced apart from the first articulating assembly.

12. The catheter system of claim 11 wherein the second articulating assembly comprises a second distal ring attached to a second proximal catheter and a second proximal ring, having a wedge shaped cross section and attached to a third proximal catheter, wherein the second proximal catheter and the third proximal catheter each have a proximal section, a distal section, and a central lumen passing therethrough, the second proximal catheter and the third proximal catheter concentrically arranged about the inner catheter and the proximal catheter.

13. The catheter system of claim 12 wherein the second distal ring has a wedge shaped cross section.

14. The catheter system of claim 12 wherein one of the second distal ring or the second proximal ring is made from a magnet and the other of the distal ring or proximal ring is made from a material that is attracted to a magnet.

15. The catheter of claim 12 wherein both the second distal ring and the second proximal ring are made from magnets.

16. The catheter system of claim 12 further comprising at least one radiopaque marker on the second distal ring and at least one radiopaque marker on the second proximal ring.

17. A method for navigating a catheter through a patient's vascular system comprising:
providing a catheter system, the catheter system comprising:
an elongate, flexible, generally tubular proximal catheter, an elongate, flexible generally tubular inner catheter, the inner and proximal catheters each having a proximal section, a distal section, and a central lumen passing therethrough, the inner catheter disposed within the central lumen of the proximal catheter,
a conical tip attached to the distal end of the inner catheter; and
at least one articulating subassembly comprising:
a distal ring attached to the inner catheter adjacent a proximal end of the conical tip; and
a proximal ring attached to the distal end of the proximal catheter, wherein at least one of the distal ring and the proximal ring has a wedge shaped cross section, at least one of the distal ring and proximal ring is made from a magnet and the other of the distal ring and proximal ring is made from material that is attracted to a magnet, and the distal ring and proximal ring are rotatable relative to each other;
navigating the distal end of the catheter through a patient's vascular system;
determining a change in direction of the distal end of the catheter; and
manipulating the at least one articulating assembly so that the distal ring and the proximal ring are rotated relative to each other and the angle of the distal tip relative to a long axis of the catheter has been altered.

18. The method of claim 17 further comprising:
manipulating the altered at least one articulating assembly into a straight line navigating configuration based on the change of direction.
